SEQUENCE LISTING (6 sequences)

SEQ ID NO: 1 - protein, 114 aa, Homo sapiens
  SIGNAL (1)..(36)
  CHAIN (37)..(114): ENA-78(1-78)
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 2 - DNA, 23 bp, Artificial
  Synthetic oligonuclotide primer
aaggccgcccgggaactgctgac

SEQ ID NO: 3 - DNA, 24 bp, Artificial
  Synthetic oligonuclotide primer
accacctgcttgcggaccctgata

SEQ ID NO: 4 - DNA, 19 bp, Artificial
  Synthetic oligonuclotide primer
catcgccagcgctggtcct

SEQ ID NO: 5 - DNA, 23 bp, Artificial
  Synthetic oligonuclotide primer
gggatgaactccttgcgtggtct

SEQ ID NO: 6 - DNA, 58 bp, Artificial
  Synthetic oligonuclotide sequence coding for shRNA
ccgggatcagtaatctgcaagtgttctcgagaacacttgcagattactgatctttttg

(19) United States
(12) Patent Application Publication
(10) Pub. No.: US 2015/0125863 A1
(43) Pub. Date: May 7, 2015

(54) CXCL5 AS A MARKER OF HORMONE ESCAPE IN PROSTATE CANCER

(71) Applicant: Institut National de la Sante Recherche Medicale (INSERM), Paris (FR)

(72) Inventors: Gwendal LAZENNEC, Montpellier Cedex 5 (FR); David VINDRIEUX, Montpellier Cedex 5 (FR); Brigitte MADLY, Montpellier Cedex 5 (FR)

(21) Appl. No.: 14/596,659

(22) Filed: Jan. 14, 2015

Related U.S. Application Data
(63) Continuation of application No. 13/641,905, filed on Jan. 2, 2013, now abandoned.

(30) Foreign Application Priority Data
Apr. 19, 2010 (FR) 10305402.9

(51) Int. Cl. G01N 33/574; C12Q 1/68

(52) U.S. Cl. CPC G01N 33/57434; C12Q 1/6886; G01N 2333/522; C12Q 2600/158; G01N 2800/52
USPC 435/6.11; 436/501; 435/7.92; 435/7.1; 435/6.12

(57) ABSTRACT

The invention relates to the use of CXCL5 as a marker for assessing hormone escape in prostate cancer cells. The invention further provides diagnostic methods and kits for assessing hormone escape in prostate cancer cells, and CXCL5 antagonists for use in the treatment or prevention of prostate cancer and/or hormone escape in prostate cancer.

The invention relates to the use of CXCL5 as a marker for assessing hormone escape in prostate cancer cells. The invention further provides diagnostic methods and kits for assessing hormone escape in prostate cancer cells, and CXCL5 antagonists for use in the treatment or prevention of prostate cancer and/or hormone escape in prostate cancer.

Prostate cancer is a disease causing significant morbidity and mortality throughout the world. The most prevalent form is prostatic adenocarcinoma. Only in the US, about 219,000 new cases of prostatic adenocarcinoma and about 27,000 deaths due to prostatic adenocarcinoma occur each year. Virtually all of these deaths from prostatic adenocarcinoma will occur in men with hormone-resistant (androgen-independent) disease.

Hormone escape is the main issue in prostate cancer treatment, as it arises for most patients treated with anti-androgens after a certain time ranging from fourteen to thirty months.

Indeed, prostate cancer is a malignancy that develops and progresses under the influence of androgenic steroids. Various forms of androgen depletion therapies are used to treat patients diagnosed with prostate cancer for which surgery is no longer an effective treatment option. The effectiveness of androgen depletion therapy for prostate cancer patients is based upon its ability to suppress proliferation of the tumor cells and to induce apoptosis of at least a fraction of these cells. Inevitably, however, residual prostate tumor cells that survive androgen depletion therapy progress to a state where they are considered to be hormone-refractory because their growth and survival is no longer suppressed in the androgen depleted environment of the treated patient. In other words, an hormone escape has occurred in these cells. The occurrence of hormone escape is linked with the high morbidity and mortality of prostate cancer.

Araki et al. (2007 Cancer Res. 67:6854-62) teaches that IL-8 (CXCL8) is a molecular determinant of androgen-independent prostate cancer growth and progression, and suggests that IL-8 antagonists may be used in the frame of chemotherapy for treating androgen-independent prostate cancer. However, Araki et al. (2007) neither teaches nor suggests that this conclusion could eventually be extended to other members of the interleukin family.

Begley et al. (2008 Neoplasia. 10:244-54) teaches that CXCL5 expression increases concordantly with prostate tumor progression. However, Begley et al. (2008) does not teach whether CXCL5 expression is a cause or a consequence of androgen dependency. In particular, Begley et al. (2008) neither teaches nor suggests that CXCL5 plays a role in the onset of hormone escape. To the contrary, Begley et al. (2008) suggests that androgen-independent mechanisms are involved in the CXCL5-mediated proliferative response of prostate cancer cells.

There is therefore a need for assessing the onset of hormone escape in patients suffering from prostate cancer, and identifying drugs which are efficient for treating patients in whom hormone escape has taken place.

### DESCRIPTION OF THE INVENTION

It has been found that the CXCL5 chemokine is highly expressed in epithelial cells of high grade prostate tumors. Moreover, invasive prostate cancer cell lines produce higher levels of CXCL5 than less aggressive prostate cancer cell lines. It has further been shown that this higher expression is due to a higher transcriptional activity of CXCL5 gene promoter.

Studies have been carried out with the androgen-receptor (AR)-positive prostate cancer cell line CWR-R1. CWR-R1 cells were transfected with the CXCL5 cDNA, and clones stably expressing CXCL5 cDNA were isolated. It was surprisingly found that expression of CXCL5 increased cell growth in vitro and reduced the ability of the cells to adhere to plastic dishes. In addition, CXCL5 expression conferred to these cells a spectacular growth in vivo.

It was surprisingly found that CXCL5 gene is androgen-regulated. Indeed, removal of androgen up-regulated its expression, whereas addition of androgens reduced its expression. In addition, treatment of CWR-R1 cells with the anti-androgen bicalutamide led to an increased CXCL5 expression.

CXCL5 treatment of CWR-R1 cells led to increased in vitro cell growth even in the presence of bicalutamide. This was confirmed in vivo. Indeed, wild-type CWR-R1 cells fail to grow in the absence of androgens. In contrast to this, the presence of CXCL5 enabled tumor take of CWR-R1 cells. Moreover, when animals bearing CWR-R1-CXCL5 tumors were castrated, hormone escape arised, whereas wild-type CWR-R1 cells failed to undergo hormone escape.

It was further found that seric CXCL5 levels are in agreement with the ones of tumor levels, demonstrating that in the frame of the present invention, CXCL5 levels can be measured in serum rather than in prostate cells. In humans, CXCL5 could also be detected in the serum and urine of patients.

Moreover, it was found that blocking CXCL5 activity strongly reduces its mitogenic effects.

In summary, it has been found that CXCL5 is an androgen-regulated genes involved in hormone escape of prostate cancer cells. CXCL5 thus constitutes a marker for hormone escape in prostate cancer, and a therapeutic target for the treatment of prostate cancer.

### DEFINITIONS

The term “CXCL5” refers to the C-X-C motif chemokine 5. The amino acid sequence of human CXCL5 is shown as SEQ ID NO: 1 (Swiss-Prot accession number P42830). The term “CXCL5” encompasses the protein of SEQ ID NO: 1 (full-length and mature isoforms) as well as homologues in other species, variants obtained by proteolytic processing, splice variants and allelic variants thereof.

As used herein, the term “prostate cancer” refers to any type of malignant (i.e. non benign) tumor located in prostatic tissues, such as e.g. prostatic adenocarcinoma, prostatic sarcoma, undifferentiated prostate cancer, prostatic squamous cell carcinoma, prostatic ductal transitional carcinoma and prostatic intraepithelial neoplasia. The prostate cancer preferably corresponds to an adenocarcinoma of the prostate.

The prostate cancer preferably corresponds to an “androgen-independent prostate cancer”, i.e. a prostate cancer which is clinically defined as hormone refractory and unresponsive.

By “method of treating” is meant a method aiming at curing, improving the condition and/or extending the lifespan of an individual suffering from a disease.

By “method of preventing” is meant a method aiming at preventing the occurrence of a disease.

The term “biological sample” refers to any type of biological sample. The biological sample may e.g. correspond to prostate tissue or to prostate cells, most preferably epithelial prostate cancer cells, which can for example be obtained by surgical excision or by biopsy. Since chemokines are secreted, they can also be directly detected in biological fluids. For instance, CXCL5 can be detected in the serum and urine of patients (see example 9). Therefore, the biological sample preferably corresponds to a biological fluid such as blood, plasma, serum, urine, semen or lymphatic fluid. In particular, as shown in example 7, seric CXCL5 level is correlated to intra-tumor expression of CXCL5. Moreover, data from the prior art confirm that it is possible to detect variations of CXCL5 expression level in the serum or plasma of patients suffering from prostatic cancer (Sung et al. Cancer Res. 2008 Dec. 1; 68(23):9996-10003, et Macoska et al. Prostate. 2008 Mar. 1; 68(4):442-52). Thus, the biological sample most preferably corresponds to plasma or serum.

It may be noted that cells derived from the prostate are found in small numbers in such biological fluids. Thus the biological fluid may optionally be enriched for prostate-derived tissue or cells. Enrichment for prostate cells may be achieved using, for example, cell sorting methods such as fluorescent activated cell sorting (FACS) using a prostate-selective antibody such as one directed to prostate-specific antigen (PSA) or prostate specific membrane antigen (PSMA). Alternatively, enrichment may be achieved using magnetic beads or other solid supports, for example a column, coated with such a prostate-specific antibody, for example an anti-PSA antibody.

“Antibody” is meant to include not only whole immunoglobulin molecules but also fragments thereof such as Fab, F(ab′)2, Fv and other fragments thereof that retain the antigen-binding site. The term “antibody” also includes genetically engineered derivatives of antibodies such as single chain Fv molecules (scFv) and single domain antibodies (dAbs). The term further includes antibody-like molecules which may be produced using phage-display techniques or other random selection techniques for molecules. The term includes all classes of antibodies and more specifically IgGs, IgAs, IgMs, IgDs and IgEs. Although the antibody may be a polyclonal antibody, it is preferred if it is a monoclonal antibody. A “monoclonal antibody” refers to an antibody that recognizes only one type of antigen. The term “monoclonal antibody” encompasses both antibodies produced by hybridomas (Kohler and Milstein 1975 Nature 256:495-7) and recombinant antibodies obtained through genetic engineering. More specifically, monoclonal antibodies encompass chimeric antibodies (Boulianne et al. 1984 Nature 312:643-6), humanized antibodies (Jones et al. 1986 Nature 321:522-5) and fully human antibodies which may be produced e.g. by phage display (Vaughan et al. 1998 Nat. Biotechnol. 16:535-9) or transgenic technology (Lonberg 2005 Nat. Biotechnol. 23:1117-25). If the antibody is going to be administered to a human patient, it is preferred if the monoclonal antibody is a fully human monoclonal antibody or a humanized monoclonal antibody.

“Antibody fragments” comprise a portion of an intact antibody, preferably the antigen binding or variable region of the intact antibody. Examples of antibody fragments include Fv, Fab, F(ab′)2, Fab′, dsFv, scFv, sc(Fv)₂, diabodies and multispecific antibodies formed from antibody fragments. The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. The human scFv fragment of the invention includes CDRs that are held in appropriate conformation, preferably by using gene recombination techniques.

“dsFv” is a VH::VL heterodimer stabilised by a disulphide bond. Divalent and multivalent antibody fragments can form either spontaneously by association of monovalent scFvs, or can be generated by coupling monovalent scFvs by a peptide linker, such as divalent sc(Fv)₂.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

The term “siRNA” refers to a small interfering RNA, which acts to degrade mRNA sequences homologous to either of the RNA strands in the duplex and can cause post-transcriptional silencing of specific genes in cells, for example, mammalian cells (including human cells) and in the body, for example, mammalian bodies (including humans). The phenomenon of RNA interference is described and discussed e.g. in Bass (2001 Nature 411:428-29), Elbahir et al. (2001 Nature 411: 494-98), Fire et al. (1998 Nature 391:806-11) and WO 01/75164, where methods of making interfering RNA also are also discussed. The siRNAs based upon the sequences and nucleic acids encoding the gene products disclosed herein typically have fewer than 100 base pairs and can be, e.g., about 30 bps or shorter, and can be made by approaches known in the art, including the use of complementary DNA strands or synthetic approaches. The siRNAs are capable of causing interference and can cause post-transcriptional silencing of specific genes in cells, for example, mammalian cells (including human cells) and in the body, for example, mammalian bodies (including humans). Exemplary siRNAs according to the present invention can have a length up to 30, 25, 22, 20, 15, 10 or 5 nucleotides, or any integer thereabout or there between. Tools for designing optimal inhibitory siRNAs include that available from DNAengine Inc. (Seattle, Wash., USA) and Ambion, Inc. (Austin, Tex., USA).

The term “shRNA” (or “short hairpin RNA”) refers to a sequence of RNA that makes a tight hairpin turn and that is used to silence gene expression via RNA interference. ShRNA are introduced into the cell by means of a viral vector, generally a lentiviral vector so that they usually get integrated into the genome of the cell. Therefore, they pass on to daughter cells allowing the gene silencing to be inherited. ShRNA constructs generally comprise a promoter to ensure that the RNA is synthesized. Once produced, the shRNA hairpin structure is cleaved by the cellular machinery into a siRNA which may then silence gene expression according to the above described mechanism.

Although having distinct meanings, the terms “comprising”, “having”, “containing’ and “consisting of” have been used interchangeably throughout this specification and may be replaced with one another.

Diagnostic Methods and Uses According to the Invention

It has been found that the CXCL5 chemokine is highly expressed in epithelial cells of high grade prostate tumors. In addition, it has surprisingly been found that CXCL5 is an androgen-regulated gene involved in hormone escape of prostate cancer cells. Finally, it was demonstrated in vivo in mice that over-expression of CXCL5 in prostate cancer cells induces hormone escape. Overall, these data show that CXCL5, which is regulated by androgens and the over-expression of which leads to hormone escape, plays a crucial role in hormone escape in prostate cancer.

Therefore, an aspect of the invention is directed to the use of CXCL5 as a marker for assessing hormone escape in prostate cancer. Increased amounts and/or expression levels of CXCL5 are indicative of hormone escape.

By “use as a marker” is meant an in vitro use, wherein CXCL5 is detected e.g. using ligands, antibodies, probes and/or primers. Therefore, the invention is also directed to the use of means for detecting CXCL5 in a biological sample for assessing hormone escape in prostate cancer. Said biological sample has previously been taken from an individual suffering from, or susceptible of suffering from, prostate cancer.

In one embodiment, CXCL5 is used in combination with at least one other chemokine such as, e.g., CXCL8.

The invention is further directed to an in vitro method for diagnosing androgen-independent prostate cancer, comprising the steps of:
a) measuring the amount and/or expression level of CXCL5 in a biological sample of an individual suffering from, or likely to suffer from, prostate cancer;
b) comparing the amount and/or expression level measured at step (a) with the amount and/or level measured in a negative control sample; and
   wherein the detection of an increase of the CXCL5 amount and/or expression level in said biological sample compared with the CXCL5 amount and/or expression level in said negative control sample indicates that said individual is likely to suffer from androgen-independent prostate cancer.

Since CXCL5 expression is correlated with hormone escape, the invention is also directed to an in vitro method for assessing hormone escape in a patient suffering from prostate cancer, comprising the steps of:
a) measuring the amount and/or expression level of CXCL5 in a biological sample of said patient; and
b) comparing the amount and/or level measured at step (a) with the amount and/or level of CXCL5 measured in a negative control sample;
   wherein the detection of an increase of the CXCL5 amount and/or expression level in said biological sample compared with the CXCL5 amount and/or expression level in said negative control sample indicates that prostate cancer cells are undergoing hormone escape, thereby assessing hormone escape in said patient.

Said increase (if any) is preferably statistically significant. The increase is considered to be statistically significant if the amount and/or expression level of CXCL5 in the biological sample is increased of at least 50%, preferably at least 2, 3, 4, 5, 6, 7, 8, 9 or 10 fold compared with the amount and/or level in the negative control sample.

In a specific embodiment, the method according to the invention comprises the steps of:
a) measuring the amount and/or expression level of CXCL5 in a biological sample of said patient; and
b) comparing the amount and/or expression level of CXCL5 measured at step (a) with the amount and/or expression level of CXCL5 in a negative control sample;
c) determining whether the amount and/or expression level of CXCL5 in said biological sample is increased compared to the amount and/or expression level of CXCL5 in said negative control sample;
   wherein an increased amount and/or expression level indicates that prostate cancer cells are undergoing hormone escape, thereby assessing hormone escape in said patient.

As used throughout the present specification, the term “control sample” refers to a sample comprising a known amount of CXCL5, or to a sample taken from an individual known to be healthy or known to be suffering from prostate cancer. Such control samples can for example comprise a known amount of CXCL5 corresponding to the mean amount of CXCL5 that has been determined in a group of individuals. CXCL5 may be present within the control sample e.g. as a polypeptide, a DNA, a RNA, a cDNA or a mRNA.

The control sample may either correspond to a positive control sample or to a negative control sample.

A “positive control sample” may for example comprise an amount of CXCL5 that is representative of the amount of CXCL5 found in individuals suffering from androgen-independent prostate cancer.

A “negative control sample” may for example comprise an amount of CXCL5 that is representative either of the amount of CXCL5 found in healthy individuals (i.e. an individual who does not suffer from prostate cancer), or of the amount of CXCL5 in individuals suffering from androgen-dependent prostate cancer.

Methods for measuring the amount and/or expression level of CXCL5 are well-known in the art. The amount and/or expression level may be measured either by quantifying mRNAs, or by quantifying proteins. Suitable methods include, e.g., immunochemistry, Elisa, Western blotting, flow cytometry, Northern blotting, PCR (e.g. RT-PCR), ligase chain reaction (LCR), transcription-mediated amplification (TMA), strand displacement amplification (SDA) and nucleic acid sequence based amplification (NASBA).

In a preferred embodiment, the amount and/or expression level of CXCL5 is measured by Elisa, e.g. through a cheminoluminescent Elisa assay. This assay may for example be carried out as described in Example 1, paragraph 15. Briefly, this assay may comprise the steps of:
coating a microtiter plates with an anti-CXCL5 antibody (e.g. polyclonal anti-human CXCL5 Ab such as MAB254 from R&D Systems);
washing said microtiter plates;
adding the biological sample;
washing the microtiter plates;
adding a biotinylated anti-CXCL5 antibody (e.g. a biotinylated polyclonal anti-human CXCL5 Ab such as BAF254 from R&D Systems)
washing said microtiter plates;
adding streptavidin-phosphatase alkaline (e.g. from BD Pharmingen);
washing the microtiter plates;
adding a luminescent substrate (e.g. the CSPD® 1,2-dioxetane luminescence substrate); and
reading luminescence (e.g. on a Centro LB960 Berthold luminometer).

The above methods according to the invention may further comprise the step of designing a treatment regimen for said individual. The choice of the suitable treatment regimen is based on the amount and/or level measured at step (a), which is indicative of the androgen-dependency of the prostate cancer. If the prostate cancer is androgen-dependent, the clinician may opt for an androgen therapy. On the other hand, if the prostate cancer is androgen-independent, the clinician may opt for a chemotherapy. These therapies may additionally be combined with radiation therapy and/or surgery.

High levels of CXCL5 indicate that the prostate cancer is severe. Therefore, patient having such cancer cells needs to be treated by an aggressive chemotherapy. The invention is thus directed to a method for selecting a patient suffering of a prostate cancer suitable to be treated by an aggressive chemotherapy comprising the steps of measuring the amount and/or expression level of CXCL5 in a biological sample from said patient, and selecting the patient if a high amount and/or expression level of CXCL5 is measured at step (a).

By “patient having a high amount and/or expression level of CXCL5” is meant a patient having an amount and/or expression level of CXCL5 that is at least 50% higher, and preferably at least 2, 3, 4, 5, 6, 7, 8, 9 or 10 times higher, than the amount and/or expression level of CXCL5 in a negative control sample.

By “aggressive chemotherapy” is meant a chemotherapy adapted for treating aggressive cancers. Specifically, such aggressive chemotherapies are second-line treatments that may induce side effects and do therefore not constitute the preferred treatment regimen in the case of a non-aggressive cancer. An aggressive chemotherapy typically corresponds to a combination chemotherapy carried out with high doses of drugs. Such second-line treatments include chemotherapies carried out with drugs such as cyclophosphamide (Astra Medica), 5-fluorouracil (Schering Health Care, Cambridge Laboratories), vincristine (Eli Lilly), cisplatin and epirubicin (Pharmacia), estramustine phosphate (Pharmacia, Pierre Fabre), vinorelbine (Pierre Fabre), paclitaxel (Bristol-Myers Squibb) and docetaxel (Sanofi-Aventis).

The above methods according to the invention may be repeated at least at two different points in time in order to monitor hormone escape in a patient and/or to monitor responsiveness of a patient to a treatment. For example, in the frame of a long-term treatment of the patient, biological samples may be taken from the patient at regular intervals (e.g. each month, every two months or twice a year).

In the frame of monitoring of a patient's responsiveness to a treatment, the biological samples are preferably taken before and after onset of the treatment of the patient.

More specifically, the invention relates to an in vitro method for monitoring responsiveness of a patient suffering from prostate cancer to a drug, said method comprises the steps of:
a) measuring the amount and/or expression level of CXCL5 in a biological sample of said patient before and after onset of a treatment of said patient with said drug;
b) comparing the amounts and/or expression levels measured at step (a); and, optionally,
c) correlating a difference in said amounts and/or expression levels with the effectiveness of the drug for treating said patient.

An increase in the amounts and/or expression levels after onset of the treatment compared with the amounts and/or expression levels before onset of the treatment indicates that hormone escape is occurring, and that said drug is not effective for treating said patient. Conversely, if no significant difference in the amounts and/or expression levels is found at step (b), or if a decrease is found at step (b), the patient responds to said drug and the drug is effective for treating said patient.

### Kits According to the Invention

The invention further discloses kits that are useful in the above methods. Such kits comprise means for detecting the amount and/or expression level of CXCL5.

They can be used, e.g. for diagnosing androgen-independent prostate cancer, for assessing hormone escape in a patient suffering from prostate cancer, for designing a treatment regimen, for monitoring the progression of prostate cancer and/or for monitoring the onset of hormone escape in a patient suffering from prostate cancer, for monitoring responsiveness to a drug and/or for adjusting the treatment of a patient suffering from prostate cancer.

The kit may further comprise means for detecting the amount and/or expression level of other chemokines than CXCL5, such as e.g. means for detecting the amount and/or expression level of CXCL8.

In a preferred embodiment, the kit according to the invention comprises, in addition to the means for detecting the amount and/or expression level of CXCL5, a control sample indicative of the amount and/or expression level of CXCL5 in an individual suffering from prostate cancer.

The kits according to the invention may for example comprise, in addition to the means for detecting the amount and/or expression level of CXCL5, a of (i) to (iv) below:
i. a positive control sample indicative of the amount and/or expression level of CXCL5 in an individual suffering from an androgen-independent cancer;
ii. a negative control sample indicative of the amount and/or expression level of CXCL5 in a healthy individual;
iii. a negative control sample indicative of the amount and/or expression level of CXCL5 in an individual suffering from an androgen-dependent cancer; and/or
iv. instructions for the use of said kit in diagnosing prostate cancer, in assessing the severity of a prostate cancer and/or in assessing the onset of hormone escape in prostate cancer.

Such a kit may for example comprise (i) and (ii), (i) and (iii), (i) and (iv), (ii) and (iii), (ii) and (iv), (iii) and (iv), (i), (ii) and (iii), (i), (ii) and (iv), (ii), (iii) and (iv) or all of (i) to (iv).

Means for detecting the amount and/or expression level of CXCL5 are well-known in the art. They include, e.g. primers and probes comprising or consisting of a fragment of the gene or cDNA encoding CXCL5 or a sequence complementary thereto, and antibodies specifically binding to CXCL5.

Such means can be labeled with detectable compound such as fluorophores or radioactive compounds. For example, the probe or the antibody specifically binding to CXCL5 may be labeled with a detectable compound. Alternatively, when the kit comprises a antibody, the kit may further comprise a secondary antibody, labeled with a detectable compound, which binds to an unlabelled antibody specifically binding to CXCL5.

The means for detecting the amount and/or expression level of CXCL5 may also include reagents such as e.g. reaction, hybridization and/or washing buffers. The means may be present, e.g., in vials or microtiter plates, or be attached to a solid support such as a microarray as can be the case for primers and probes.

The kit may for example include primers of SEQ ID Nos. 4 and 5 as a mean for detecting the amount and/or expression level of CXCL5. Alternatively, the kit may include the MAB254 antibody (R&D Systems, Minneapolis, USA) and/or the AF254 antibody (R&D Systems, Minneapolis, USA) as a mean for detecting the amount and/or expression level of CXCL5.

In a specific embodiment, the kit is suitable for performing a cheminoluminescent Elisa assay, such as e.g. the cheminoluminescent Elisa assay described in Example 1, paragraph 15. Such a kit may for example comprise, as means for detecting the amount and/or expression level of CXCL5:
an anti-CXCL5 antibody (e.g. polyclonal anti-human CXCL5 Ab such as MAB254 from R&D Systems), optionally coated on a microtiter plate;
a biotinylated anti-CXCL5 antibody (e.g. a biotinylated polyclonal anti-human CXCL5 Ab such as BAF254 from R&D Systems); optionally
streptavidin-phosphatase alkaline (e.g. from BD Pharmingen); and optionally
a luminescent substrate (e.g. the CSPD® 1,2-dioxetane luminescence substrate).

In Vivo Imaging

CXCL5 levels in the prostate may also be detected in vivo using imaging techniques. Therefore, a further aspect of the invention provides a diagnostic agent comprising a detectable moiety and an antibody specifically binding to CXCL5 for use in imaging of androgen-independent prostate cancer.

As presented hereabove, expression levels of CXCL5 are positively correlated with the invasiveness and androgen dependency of prostate cancer cells. Therefore, such diagnostic agents are useful for diagnosing androgen-independent prostate cancer, for assessing hormone escape in a patient, for designing a treatment regimen, for monitoring the progression of prostate cancer and/or for monitoring the onset of hormone escape in a patient suffering from prostate cancer, for monitoring responsiveness to a drug and/or for adjusting the treatment of a patient suffering from prostate cancer.

Imaging techniques include, e.g., scintigraphic studies, magnetic resonance imaging (MRI), optical imaging, single photon emission computed tomography (SPECT) and positron emission tomography (PET).

By “detectable moiety” is meant a moiety which, when located at the target site following administration of the diagnostic agent of the invention to a patient, may be detected non-invasively from outside the body. The detectable moiety depends on the chosen imaging technique. Typically, the readily detectable moiety is or comprises a radioactive atom.

For scintigraphic studies, suitable detectable moieties include e.g. technetium ^{99m}Tc, ¹²³I, ¹¹C, ¹³N, ¹⁵O, ¹⁸F, ⁵¹Cr, ⁶⁷Ga, ¹¹¹In, ^{113m}In, ¹³¹I, ¹³³Xe and ²⁰¹Tl. For MRI, suitable detectable moieties include, for example, such as ¹²⁵I, ¹²³I ¹³¹I, ¹¹¹In, ¹⁹F, ¹³C, ¹⁵N, ¹⁷O, gadolinium, manganese and iron. For optical imaging, suitable detectable moieties include a number of near-infrared (NIR) fluorophores such as Kodak X-SIGHT Nanospheres as well as dyes and dye conjugates such as Cy®5.5, Cy7, Alexa Fluor® 680, Alexa Fluor 750, IRDye® 680, and IRDye 800CW. In the frame of SPECT, ¹⁸F and radiotracers such as ^{99m}Tc, ¹¹¹In, ¹²³I, ²⁰¹Tl and ¹³³Xe can be used as detectable moieties. Detectable moieties suitable for performing PET include e.g. ¹¹C, ¹³N, ¹⁵O, ¹⁸F, ⁶⁴Cu, ⁶²Cu, ¹²⁴I, ⁷⁶Br, ⁸²Rb and ⁶⁵Ga.

Preferably, the readily detectable moiety comprises or consists of technetium-99m (^{99m}Tc), ¹²³I, ¹²⁵I, ¹³¹I, ¹⁸F or a NIR fluorophore.

A further aspect of the invention provides a method of imaging prostate cancer comprising the steps of (i) administering the diagnostic agent according to the invention to an individual suffering from, or likely to suffer from, prostate cancer, and (ii) detecting the presence of absence of binding of said diagnostic agent to the prostate of said individual.

A further aspect of the invention provides a method of diagnosing prostate cancer, said method comprising the steps of (i) administering the diagnostic agent according to the invention to an individual suffering from, or likely to suffer from, prostate cancer, and (ii) detecting the presence of absence of binding of said diagnostic agent to the prostate of said individual, wherein the detection of binding of said diagnostic agent to the prostate of said individual indicates that said individual is likely to suffer from prostate cancer.

Said method can be also be used for assessing the severity of prostate cancer and/or for assessing the onset of hormone escape in prostate cancer by detecting the level of binding of said diagnostic agent to the prostate of said individual, and correlating said level with severity of prostate cancer and/or onset of hormone escape in prostate cancer.

Antagonists According to the Invention and Methods for Screening for Such Antagonists

It has surprisingly been found that in prostate cancer, CXCL5 levels are negatively regulated by androgens (see FIG. 5) and positively regulated by anti-androgens (see FIG. 6). On the other hand, removal of androgens increases CXCL5 expression. This latter phenomenon is very similar to the hormone escape phenomenon which is observed in patients suffering from prostate cancer. Therefore, it is believed that upon hormone therapy treatment, CXCL5 levels are increased, thereby rendering prostate cancer cells more aggressive, more metastatic and capable of proliferating in the absence of androgens. As a consequence, hormone escape may be prevented or treated by inhibiting CXCL5 biological activity.

Therefore, the invention provides a CXCL5 antagonist for use in the treatment or prevention of prostate cancer and/or of hormone escape in prostate cancer. The CXCL5 antagonist according to the invention is preferably for use in the treatment or prevention of an androgen-independent prostate cancer.

As used herein, the term “CXCL5 antagonist” refers to refers to a compound that inhibits or reduces the biological activity of CXCL5.

The biological activity of CXCL5 depends both on its concentration (i.e. its expression level) and on its specific activity. Therefore, the CXCL5 antagonist in accordance with the invention may for example reduce or inhibit (i) CXCL5 expression in prostatic cells and/or (ii) binding of CXCL5 to a binding partner, thereby reducing or inhibiting signal transmission within the signaling pathway.

Methods for determining whether a compound is a CXCL5 antagonist are well-known by the skilled in the art.

For example, the skilled in the art can assess whether a compound reduces or abolishes CXCL5 expression in prostatic cells by RT-PCR, Northern Blotting, ELISA, immunostaining or Western Blotting. The protocols provided in Example 1.6 and 1.7. may for example be used.

The biological activity of CXCL5 may also be measured by assessing the capacity of CXCL5 to bind to its natural binding partners in prostatic cells such as e.g. its receptor(s). For example, CXCL5 is known to bind to the CXCR2 receptor. Binding of CXCL5 to one of its binding partners (e.g. CXCR2) may for example be assessed using a two hybrid system, immunoprecipitation or a surface plasmon resonance equipment (BIAcore). A compound reducing or abolishing binding of CXCL5 to at least one of its natural binding partners (e.g. CXCR2) in prostatic cells is defined as a CXCL5 antagonist.

Alternatively, the biological activity of CXCL5 may be assessed by measuring chemotaxis. A compound reducing or abolishing the capacity of CXCL5 to provoke chemotaxis is defined as a CXCL5 antagonist.

CXCL5 biological activity can also be measured by determining whether the signaling cascade induced by CXCL5 is activated. For example, quantification of ERK phosphorylation by Western Blot can be measured (see e.g. Begley et al. 2008 Neoplasia 10:244-254)

The CXCL5 antagonist may correspond to any type of compound. It may for example correspond to a dominant negative mutant of CXCL5, a chemical compound such as a small molecule, an antisense RNA, an interfering RNA (e.g. a siRNA or a shRNA), an aptamer, a peptide or an antibody.

The CXCL5 antagonist according to the invention is preferably purified and/or isolated, i.e. it is purified and/or isolated from human body, from animal body, and/or from a library of compounds. In a specific embodiment, the CXCL5 antagonist according to the invention does not correspond to a naturally-occurring compound. It is preferably formulated into a pharmaceutical composition.

The invention is also directed to a method of treating or preventing prostate cancer and/or of hormone escape in prostate cancer comprising the step of administering an effective amount of a CXCL5 antagonist to an individual in need thereof. By “effective amount” is meant an amount sufficient to achieve a concentration of CXCL5 antagonist which is capable of preventing or treating the disease to be treated. Such concentrations can be routinely determined by those of skilled in the art. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, etc. It will also be appreciated by those of stalled in the art that the dosage may be dependent on the stability of the administered CXCL5 antagonist. By “individual in need thereof” is meant an individual suffering from, or likely to suffer from, the disease to be treated or prevented. The individual to be treated in the frame of the invention may correspond to any mammal. In a preferred embodiment, the individual is a human.

Such CXCL5 antagonists are well-known in the art. For example, antagonists of CXCR2, and thus of CXCL5, include:
SB225002, which is a potent and selective CXCR2 chemokine receptor antagonist (White et al. 1998 J Biol Chem 273:10095-98);
SB272844 (Glynn et al. 2002 Pulm Pharmacol Ther. 15:103-10);
Repertaxin, which is a noncompetitive allosteric inhibitor of the CXCR1 and CXCR2 receptors;
SB265610, which is a high-affinity CXCR2 antagonist (de Kruijf et al. 2009 J Pharmacol Exp Ther 329:783-90);
SCH527123 and SCH479833, which are orally active small-molecule antagonists targeting CXCR2/CXCR1 (Holz et al. 2010 Eur Respir J 35:564-70 and Singh et al. 2009 Clin Cancer Res 15:2380-6);
CXCL8(3-74)K11R/G31P, which is a CXCL8 mutant and a CXCR1/CXCR2 antagonist (Gordon et al. 2005 J Leukoc Biol 78:1265-72);
compounds of the formula I as described page 4 line 22 to page 12 line 6 of patent application WO 2008/000408:

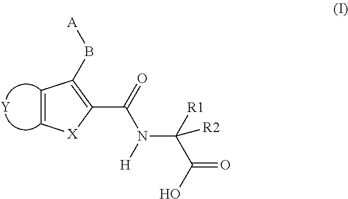

and in particular the following compounds:
2-Methyl-2-{[1-(4-trifluoromethoxy-benzyloxy)-5,6,7,8-tetrahydro-naphthale-ne-2-carbonyl]-amino}-propionic acid; 2-{[1-(Benzothiazol-2-ylmethoxy)-5,6,7,8-tetrahydro-naphthalene-2-carbonyl]-amino}-2-methyl-propionic acid; 2-Methyl-2-{[1-(4-trifluoromethyl-benzyloxy)-5,6,7,8-tetrahydro-naphthalene-2-carbonyl]-amino}-propionic acid; 2-Methyl-2-{[1-(6-trifluoromethyl-pyridin-3-ylmethoxy)-5,6,7,8-tetrahydro-naphthalene-2-carbonyl]-amino}-propionic acid; 2-{[1-(Benzothiazol-2-ylmethoxy)-5,6,7,8-tetrahydro-naphthalene-2-carbonyl]-amino}-2-methyl-butyric acid; 2-Methyl-2-{[1-(4-trifluoromethyl-benzyloxy)-5,6,7,8-tetrahydro-naphthalene-2-carbonyl]-amino}-butyric acid; 2-Methyl-2-{[1-(6-trifluoromethyl-pyridin-3-ylmethoxy)-5,6,7,8-tetrahydro-naphthalene-2-carbonyl]-amino}-butyric acid; 2-{[1-(2-Fluoro-4-trifluoromethyl-benzyloxy)-5,6,7,8-tetrahydro-naphthalene-2-carbonyl]-amino}-2-methyl-propionic acid; 2-Methyl-2-{[1-(4-trifluoromethoxy-benzyloxy)-5,6,7,8-tetrahydro-naphthalene-2-carbonyl]-amino}-butyric acid; 2-{[1-(2-Fluoro-4-trifluoromethyl-benzyloxy)-5,6,7,8-tetrahydro-naphthalene-2-carbonyl]-amino}-2-methyl-butyric acid; 2-Methyl-2-{[7-methyl-4-(4-trifluoromethyl-benzyloxy)-indane-5-carbonyl]-amino}-propionic acid; 2-Methyl-2-{[7-(4-trifluoromethyl-benzyloxy)-benzo[b]thiophene-6-carbonyl]- -amino}-propionic acid; 2-Methyl-2-{[4-(4-trifluoromethyl-benzyloxy)-benzo[b]thiophene-5-carbonyl]- -amino}-propionic acid; 2-Methyl-2-{[4-(5-trifluoromethyl-pyridin-2-ylmethoxy)-benzo[b]thiophene-5- -carbonyl]-amino}-propionic acid; and
   N,N′-diarylureas displaying selective CXCR2 antagonist function (Widdowson et al. 2004 J Med Chem 47:1319-21).

In a preferred embodiment, said CXCL5 antagonist is an antibody. Antibodies specifically recognizing CXCL5 are well-known in the art and include, e.g., the goat ENA-78 polyclonal antibody commercialized by R&D systems (Catalogue No. AF254). Thus the antibody may for example correspond to the ENA-78 polyclonal antibody commercialized by R&D systems, to a monoclonal antibody cross-reacting with that antibody, or to a monoclonal antibody obtained from that polyclonal antibody, e.g. obtained through production of hybridomas as described in Kohler and Milstein (1975 Nature 256:495-7).

In another preferred embodiment, the CXCL5 antagonist is a siRNA or a shRNA. Methods for obtaining siRNAs and shRNAs are well-known in the art. For example, tools for designing inhibitory siRNAs may be purchased from DNAengine Inc. (Seattle, Wash., USA) or Ambion, Inc. (Austin, Tex., USA). In addition, siRNAs and shRNAs targeting CXCL5 can be purchased e.g. from Sigma-Aldrich. The shRNA can for example correspond to a shRNA encoded by the sequence of SEQ ID NO: 6.

Alternatively or additionally, the skilled in the art may isolate such CXCL5 antagonists by carrying out a screening.

Therefore, another aspect of the invention is directed to an in vitro method of screening for drugs for the treatment of a prostate cancer, in particular of an androgen-independent cancer, comprising the steps of:
a) providing a test compound; and
b) determining whether said test compound inhibits the biological activity of CXCL5, for example in prostatic cells;
   wherein the determination that said test compound inhibits the biological activity of CXCL5 indicates that said test compound is a drug for the treatment or the prevention of prostate cancer.

Any method well-known in the art may be used at step (b), such as one of the methods described hereabove. In a preferred embodiment, step (b) is carried out with prostatic cells, most preferably prostate cancer cells. For example, said step of determining whether said test compound inhibits the biological activity of CXCL5 may for example comprise or consists of the step of:
determining whether said test compound inhibits CXCL5 expression in prostatic cells; and/or
determining whether said test compound inhibits binding of CXCL5 to a natural binding partner in prostatic cells.

More specifically, this in vitro method may comprise the steps of:
a) providing a test compound; and
b) determining CXCL5 biological activity in the presence of said test compound, for example in prostatic cells;
c) determining CXCL biological activity in the absence of said test compound, for example in prostatic cells; and
d) comparing the results of steps (b) and (c);
   wherein the determination that the biological activity measured at step (b) is lower than the biological activity measured at step (c) indicates that said test compound is a drug for the treatment or the prevention of prostate cancer.

The test compound may correspond to any type of compound. It may for example correspond to a dominant negative mutant of CXCL5, a small molecule, an antisense RNA, an interfering RNA (e.g. a siRNA, or a shRNA), an aptamer, a peptide and an antibody. In a preferred embodiment, a library of small molecules, peptides, antibodies or aptamers is screened with the method according to the invention.

The invention is also directed to the use of CXCL5 as a target for screening for a drug (a CXCL5 antagonist) for the treatment of prostate cancer, and to the use of CXCL5 as a target for screening for a drug (a CXCL5 antagonist) for the prevention of hormone escape in prostate cancer.

Cellular and Animal Models

Another aspect of the invention is directed to a recombinant cell line derived from a prostate cancer cell characterized in that its genome comprises an expression vector comprising a nucleic acid encoding CXCL5. Such recombinant cell lines stably express CXCL5 and can be used e.g. as cellular models for androgen-independent prostate cancer cells.

The invention further pertains to a non-human animal model for androgen-independent prostate cancer comprising the recombinant cell line according to the invention. Such a non-human animal may be used as models for androgen-independent prostate cancer, e.g., in the frame of research relating to prostate cancer and during preclinal trials of drugs for the treatment or prevention of prostate cancer.

The invention also pertains to a method for producing a non-human animal model for androgen-independent prostate cancer comprising the step of inoculating the recombinant host cell in accordance with the invention to the prostate of said animal, as well as the animal model obtainable by such a method.

The animal may correspond to any non-human animal such as e.g. a mouse, a rat, a rabbit or a monkey. In a preferred embodiment, the animal is an athymic nude mice, for example a Nu/Foxn1 athymic nude mice.

All references cited herein, including journal articles or abstracts, published patent applications, issued patents or any other references, are entirely incorporated by reference herein, including all data, tables, figures and text presented in the cited references.

The invention will be further evaluated in view of the following examples and figures.

### DESCRIPTION OF THE FIGURES

FIG. 1 shows that CXCL5 expression levels are positively correlated with Gleason scores of prostate cancer tissues. The quantification of CXCL5 was carried out by immunostaining in prostate cancer tissues having different Gleason scores. The number of patients is indicated.

FIG. 2 shows that invasive prostate cancer cell lines produce high levels of CXCL5. A. Elisa quantification of CXCL5 secretion by prostate cancer cell lines. Results represent the mean±SD of 3 independent experiments. B. Quantification of CXCL5 RNA levels in prostate cancer cell lines by real time PCR. Results represent the mean±SD of 3 independent experiments. C. Measure of CXCL5 gene promoter activity in prostate cancer cell lines. Results represent the mean±SD of 3 independent experiments.

FIG. 3 shows that CXCL5 enhances the proliferation of CWR-R1 cells and reduces their attachment ability. A. CXCL5 secretion levels in CWR-R1, CWR-R1-LUC, C2S2 and C3S2 stable clones. C2S2 and C3S2 are two CWR-R1 clones stably transfected with CXCL5 cDNA. B. In vitro proliferation of CWR-R1 cells. Results represent the mean±SEM of 3 independent experiments. C. In vitro cell adhesion of CWR-R1 cells on plastic dishes after different times. Results represent the mean±SD of 3 independent experiments.

FIG. 4 shows that CXCL5 enhances dramatically in vivo tumor growth. CWR-R1-LUC or CWR-R1C2S2 cells were injected orthotopically in the prostate of athymic mice. In vivo tumor growth was measured with a Berthold NightOwl camera. Results represent the mean±SD of 6 mice per group.

FIG. 5 shows that CXCL5 RNA levels are down-regulated by androgens in LNCaP and CWR-R1 cells. CWR-R1 (A) or LNCaP (B) cells were cultured either in the presence of either of: whole fetal calf serum (FCS), charcoal-stripped serum (CDFCS) or CDFCS supplemented with 10-8 M Dihydrotestosterone (CDFCS+DHT). CXCL5 RNA levels were measured by real-time PCR. CXCL5 levels in the presence of FCS were set to 1. Results represent the mean±SD of 3 independent experiments.

FIG. 6 shows the effect of the anti-androgen bicalutamide. A. CXCL5 RNA levels are up-regulated by the anti-androgen bicalutamide in CWR-R1 cells. CWR-R1 cells were cultured in whole fetal calf serum (FCS), in the absence or in the presence of 0.1, 1 or 10 WI of bicalutamide. CXCL5 RNA levels were measured by real-time PCR. CXCL5 levels in the presence of FCS were set to 1. Results represent the mean±SD of 3 independent experiments. B. CXCL5 enhances the proliferation of CWR-R1 cells both in the absence and in the presence of the anti-androgen bicalutamide. CWR-R1 cells were cultured in FCS. Some cells were treated with 10-5 M bicalutamide. The cells were either treated with Ethanol vehicle (C) or with CXCL5 (1 or 50 ng/ml). Proliferation was quantified 5 days after treatment by counting the cells.

FIG. 7 shows that CXCL5 confers an androgen-independent growth to prostate cancer cells in vivo. A. CWR-R1-Luc or CWR-R1-CXCL5 (clone C2S2) cells were injected in the prostate of an athymic mouse that had been castrated 6 days before injection. Tumor growth was measured every week with a Berthold NightOwl camera. Results represent the mean±SD of 8 mice per group. B. CWR-R1-Luc or CWR-R1-CXCL5 (clone C2S2) cells were injected in the prostate of athymic mice. 14 days after injection, mice were divided in two groups: one which was castrated and the other sham-treated. Tumor growth was measured every week with a Berthold NightOwl camera. Results represent the mean±SD of 8 mice per group. The arrows indicate the time of castration.

FIG. 8 shows that CXCL5 levels in serum correlate with intra-tumor expression. A. Total extracts were prepared from prostate tumors of animals implanted with CWR-R1 or CWR-R1-CXCL5 cells. CXCL5 intra-tumoral levels were determined by Elisa. Results represent the mean±SD of 5 animals. B. Serum from the same mice were collected and assayed for CXCL5 content. Results represent the mean±SD of 5 animals.

FIG. 9 shows that blocking CXCL5 inhibits PC-3 cell proliferation. A. PC-3 cells were grown for 4 days and either non treated (Control) or treated with goat serum (diluted 1/20) as isotype control antibody (Isotype) or with 0.5 μg/ml of CXCL5 antibody (CXCL5 Ab). Medium with or without treatment was changed at day 2. Results represent the mean of luciferase activity±SD after 4 days of 3 independent experiments. B, C. PC-3 cells were transfected with Non-Target shRNA Control (shCt) or shCXCL5 vector. 4 days later, the medium was collected and CXCL5 levels were measured by Elisa (B). The number of cells was counted on day 4 (C). A representative experiment is shown here.

FIG. 10 shows that CXCL5 levels can be measured in human serum and urine. CXCL5 levels were determined by chemiluminescent ELISA in the serum (A) or urine (B) of 20 patients.

### DESCRIPTION OF THE SEQUENCES

SEQ ID NO: 1 shows the amino acid sequence of CXCL5.

SEQ ID Nos: 2-5 show the sequence of primers used for detecting mRNA levels in the frame of real-time FOR experiments.

SEQ ID NO: 6 shows the sequence encoding an shRNA targeting CXCL5.

### EXAMPLES

### Example 1

### Materials and Methods

1.1. Cell Culture

CWR-R1 and LNCaP cells were maintained in RPMI 1640 with Glutamax (Invitrogen, Carlsbad, USA), completed with 10% Foetal Calf Serum (FCS) and gentamycin. For androgen stimulations, cells were weaned off steroids before any experiment by culturing them in phenol red-free RPMI 1640 supplemented with 10% CDFCS (charcoal dextran-treated FCS) for 4 days.

1.2. Immunohistochemistry of CXCL5

Tissue MicroArray (TMA) from Prostate Cancer, containing 40 pathologic scores (Gleason 6 to 10) and 8 non pathologic scores, were used (SuperBioChips Laboratories, Korea).

Immunostaining was performed on the TMA using standard avidin-biotin complex techniques and a mouse monoclonal antibody against CXCL5. The slide was pretreated by microwaving the slide in Tris buffer pH 9.0 in order to retrieve antigens. The TMA was then incubated 2 h at room temperature with primary CXCL5 antibody (Human CXCL5/ENA-78 MAB254; R&D Systems, Minneapolis, USA) at the concentration of 15 μg/ml of PBS. CXCL5 expression was scored in a blinded fashion as negative (score=0), weak (score=1), moderate (score=2), or strong (score=3) based on the intensity of staining. Product scores were calculated for all tissue cores, and the mean product scores were determined for each Gleason score.

1.3. Generation of Cell Lines that Express CXCL5 and shRNA Constructs

CWR-R1 cells were transfected with CMV-Luciferase Firefly using JetPEI reagent (MP Biomedicals, Irvine, USA) and with either pLv01-CXCL5 plasmid (Origene, Rockville, USA) or an empty vector as control. The transfection was done according to the manufacturer's protocol. Colonies were selected in 2 mg/ml G418. Luciferase activity of each colonies were tested using luminometer MITRAS after injection of Luciferase activity buffer (Promega, Madison, USA). The constitutive expression of CXCL5 was measured using ELISA.

1.4. Cell Adhesion of Cell Lines on Plastic Dishes

To study the cell adhesion of CWR-R1 stably expressing CXCL5, clones were seeded on plastic dishes. 200000 cells were seeded into a 12 wells plate. Wells were washed at different times (5, 10, 15, 30, 45, 60 minutes) with PBS (2×). 200 μl of Lysis buffer (Promega, Madison, USA) were then added in each well. The luciferase activity in each well was measured as previously described. The percentage of adhesion was calculated by comparing the luminescence at different times to the luminescence of the number of cells initially seeded in each well.

1.5. Transient Transfection CXCL5 Promoter

The CXCL5 promoter, consisting of nucleotides −1379/+43 relative to the CXCL5 start site, was cloned in the pxP2 luciferase reporter plasmid (GenBank Accession Number AF093682.1) 3.10⁵ of steroid-weaned cells were plated in 12-well plates in phenol red-free RPMI 1640 supplemented with 10% CDFCS 24 h before transfection. Transfections were performed using lipofectamine according to the manufacturer's recommendations, i.e. using 2 μg of CXCL5 promoter pxP2-CXCL5 luciferase reporter along with 0.5 μg of the internal reference reporter plasmid (CMV-Gal) per well. After 6 hours of incubation, the medium was removed and the cells were placed into fresh medium. Twenty-four hours later, cells were harvested and assayed for luciferase activity using a Centro LB960 Berthold luminometer. β-galactosidase was determined as previously described (Freund et al. 2004 Oncogene 23:6105-6114).

1.6. Elisa Quantification of CXCL5

CXCL5 secretion was measured by ELISA (R&D Systems, Minneapolis, USA), according to manufacturer recommendations. Briefly, flat bottom 96-well microtiter plates (Probind, Falcon) were coated with 4 μg/ml specific polyclonal anti-human CXCL5 Antibody (MAB254, R&D Systems) in PBS and incubated overnight at room temperature. The plates were then washed with PBS (pH 7.5) and 0.05% Tween 20 (wash buffer). Plates were blocked with 2% BSA and 5% sucrose in PBS for 1 hour at room temperature and then washed three times with wash buffer. Sample or standard was added, and the plates were incubated at room temperature for 2 hours. For intra-tumor proteins, 80 μg of total extracts were used. For seric levels of CXCL5, 50 μl of serum was used. Plates were washed three times. Biotinylated polyclonal anti-human CXCL5 Antibody (AF254, R&D Systems) was added at 600 ng/ml (in PBS pH 7.5 and 0.05% Tween 20), and plates were incubated at room temperature for 2 hours. Plates were washed three times, streptavidin-HRP conjugate was added, and the plates were incubated for 20 minutes at room temperature. Plates were washed again, and 3,3′,5,5′-tetramethylbenzidine chromogenic substrate was added. Plates were read at 450 nm in an automated microtiter plate reader.

1.7. Chemiluminescent CXCL5 ELISA

CXCL5 secretion was measured by ELISA (R&D Systems), with some modifications to use a chemiluminescent approach. Briefly, flat bottom 96-well opaque white microtiter plates (Nunc) were coated with specific polyclonal anti-human CXCL5 Ab (MAB254, R&D Systems) (2 μg/ml in PBS) overnight at 4° C. and then washed with PBS (pH 7.5) plus 0.05% Tween 20 (wash buffer). Plates were blocked with blocking buffer (0.2% casein in PBS) for 1 h at room temperature and then washed three times with wash buffer. Sample or standard were added, and the plates were incubated at room temperature for 1 h. For seric levels of CXCL5, 2 μl of serum was used, and for urine, 20 μl of samples was used. Plates were washed three times. Biotinylated polyclonal anti-human CXCL5 Ab (BAF254, R&D Systems) (100 ng/ml in blocking buffer) was added, and plates were incubated at room temperature for 1 h. Plates were washed three times, streptavidin-phosphatase alkaline (BD Pharmingen) was added, and the plates were incubated for 30 min at room temperature. Plates were washed again, and CSPD® 1,2-dioxetane luminescence substrate was added during 30 min before reading luminescence on a Centro LB960 Berthold luminometer.

1.8. Real-Time PCR Quantification of CXCL5 RNA Levels

Total RNA was prepared using TriReagent (Euromedex, Souffelweyersheim, France). The amount of RNA was estimated by spectrophotometry at 260 nm. Complementary DNAs (cDNA) were obtained from reverse transcription (RT) of 3 μg of total RNA in 13 μl of water RNase free. 1 μl of random hexanucleotides as primers (pd(N)6, 50 mM) in the presence of 1 μl of dNTPs (250 mM; Gibco BRL) were added to the RNA and incubated 5 minutes at 70° C. After, 5 μl of mix containing reverse transcriptase (1 μl) and reverse transcriptase buffer 5× (4 μl) were added to the RNA solution and incubated 1 h at 42° C. Real-time PCR quantification was then performed using a SYBR Green approach (Light Cycler; Roche) as previously described (Lucas et al. 2003 Biochem Biophys Res Commun 309:1011-1016). For each sample, CXCL5 mRNA levels were normalized with RS9 mRNA levels (reference gene). The primers used were the following.

RS9 Forward:
(SEQ ID NO: 2)
5′-AAGGCCGCCCGGGAACTGCTGAC-3′
RS9 Reverse:
(SEQ ID NO: 3)
5′-ACCACCTGCTTGCGGACCCTGATA-3′
CXCL5 Forward:
(SEQ ID NO: 4)
5′-CATCGCCAGCGCTGGTCCT-3′
CXCL5 Reverse:
(SEQ ID NO: 5)
5′-GGGATGAACTCCTTGCGTGGTCT-3′

1.9. Proliferation Assays

2.5×10⁴ cells per well were seeded in triplicate in 24-well culture plates in complete growth media previously described. Cell numbers were determined every two days for 6 days by counting with a malassez slide or by measuring of luciferase activity. For proliferation in complete fetal calf serum, cells were maintained in 1% FCS with RPMI 1640. To test the effects of androgens, cells were maintained in phenol-red free RPMI 1640 medium supplemented with 10% FCS.

1.10. Animals

Male Nu/Foxn1 athymic nude mice, 8 weeks old, were obtained from Harlan. Mice were acclimatized for 1 week before the experiment, and were kept under pathogen-free conditions in laminar-flow boxes (6 mice/cage) and maintained under standard conditions (22±2° C., 45±10% relative humidity, 12 h light/12 h dark cycle each day, standard diet and water ad libitum). All experiments were performed in accordance with the French guidelines for experimental animal studies.

1.11. Xenograft and In Vivo Bioluminescent Imaging

Before injection, CWR-R1-Luc or CWR-R1-CXCL5 cells were trypsinized. 10⁶ cells were prepared in 40 μl PBS, combined with Matrigel (3:1, v/v, BD Biosciences). One group of mice (n=16) was castrated one week before graft. All the mice were orthotopically grafted in the prostate surgically exposed of anaesthezied animal with 10⁶ of CWR-R1-Luc or CWR-R1-CXCL5 cells. The Luciferase activity was then measured weekly for 8 weeks. To measure luciferase activity, mice were first sedated by isoflurane gas anesthesia system (T.E.M., Bordeaux, France). Mice were then injected intraperitoneally with 125 mg/kg body weight of luciferin (sodium salt; Promega) in aqueous solution. Luminescence was measured using NightOWL II LB 981 CCD camera and integrated for a 5-min period. The signal intensities from regions of interest (ROI) were obtained and data were expressed as photon (counts/s). Background was defined from a region of the same size placed in a non-luminescent area nearby the animal and then subtracted from the measured luminescent signal intensity. All light measurements were performed under the same conditions, including camera settings, exposure time, distance from the animals, and region size. A pseudocolor luminescent image from blue (least intense) to red (most intense), representing the spatial distribution of the detected photons emitted within the animal, was generated using WinLight software (Berthold Technologies). For the measurement of seric CXCL5 levels, the mice were sacrificed at day 42, and blood and tumor were collected.

1.12. Protein Extracts Preparation

Tumor samples were crushed into ceramic beads-containing tubes (Lysing matrix, MP Biomedical), and twice the weight of samples of TEG buffer (10 mM Tris-HCl, pH7.4, 1.5 mM EDTA, and 10% glycerol containing protease inhibitors (5 μg/ml aprotinin, leupeptin and pepstatin A, and 0.1 mM phenylmethylsulfonylfluoride)) in a MagNA Lyser machine (Roche) at 7000 r/min for 15 seconds. The lysate was then centrifuged at 10 000 rpm for 30 minutes at 4° C., and the supernatant was saved.

1.13. Blocking Experiments with CXCL5 Antibody

10³ PC-3 cells were plated in 24 well plated in 1% FCS with RPMI 1640. The next day for 4 days and either non treated (Control) or treated with goat serum (diluted 1/20) as isotype control antibody or with 0.5 μg/ml of CXCL5 antibody (AF-254, R&D system). Medium with or without treatment was changed at day 2. Cell numbers were determined at day 4 by measuring of luciferase activity.

1.14. Blocking Experiments with shCXCL5

10⁵ PC-3 cells plated in 6 well plated in 10% FCS with RPMI 1640 were transfected with 4 μg of Non-Target shRNA Control Vector (ShC002, Mission Library, Sigma-Aldrich) or shCXCL5 vector (SHCLND-NM_{—}002994-TRCN0000057934NM_{—}002994.3-304s1c1TRC1, Sequence SEQ ID NO: 6: CCGGGATCAGTAATCTGCAAGTGTTCTCGAGAACACTTGCAGATTACTGATCTTTTTG Mission Library, Sigma-Aldrich) using Jet-PEI® protocol in each well. 8 h after transfection medium was changed. CXCL5 secretion was quantified 48 h later in conditioned medium by ELISA (Duoset, DY254, R&D system). Cells were counted 4 days after transfection using malassez slide.

1.15. Human Samples

Serum and urine were obtained from a series of random patients consulting for a suspicion of prostate cancer, with no indication whether the cancer was diagnosed or not.

### Example 2

### CXCL5 is Expressed in More Aggressive Prostate Cancers

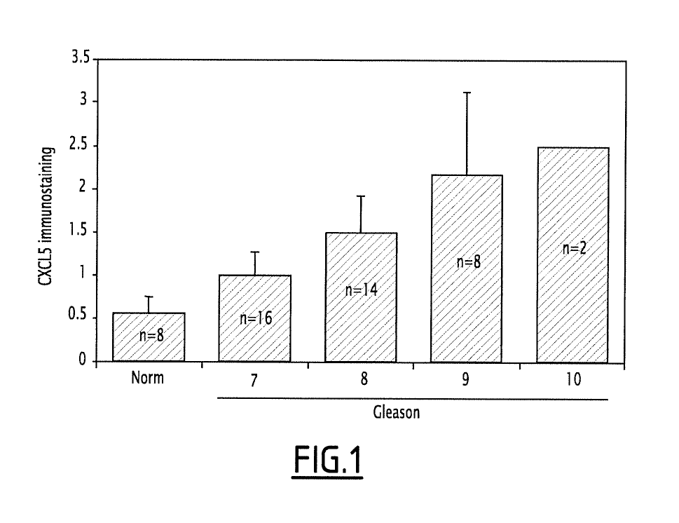

An evaluation of CXCL5 expression in normal prostate and prostate cancer was carried out by immunohistochemistry. The results showed that CXCL5 is produced mainly by epithelial prostate cancer cells. Moreover, CXCL5 levels increased with Gleason score (FIG. 1), an indicator of aggressiveness of prostate tumors.

CXCL5 secretion in distinct prostate cancer cell lines displaying distinct aggressiveness, and expressing or not androgen-receptor (AR, the mediator of androgen sensitivity of prostate cancer cells), was next studied by ELISA. PC3, PC3-LUC, DU145 and MDAPCa1 are aggressive prostate cancer cell lines which do not express AR. BRF41T, CWR-R1, 22RV1, LNCaP, C4-2, and LAPC4 are prostate cancer cells that moderately aggressive and express AR. HPV7, BPH-1, BRF-55T are benign hyperplasic prostate cell lines.

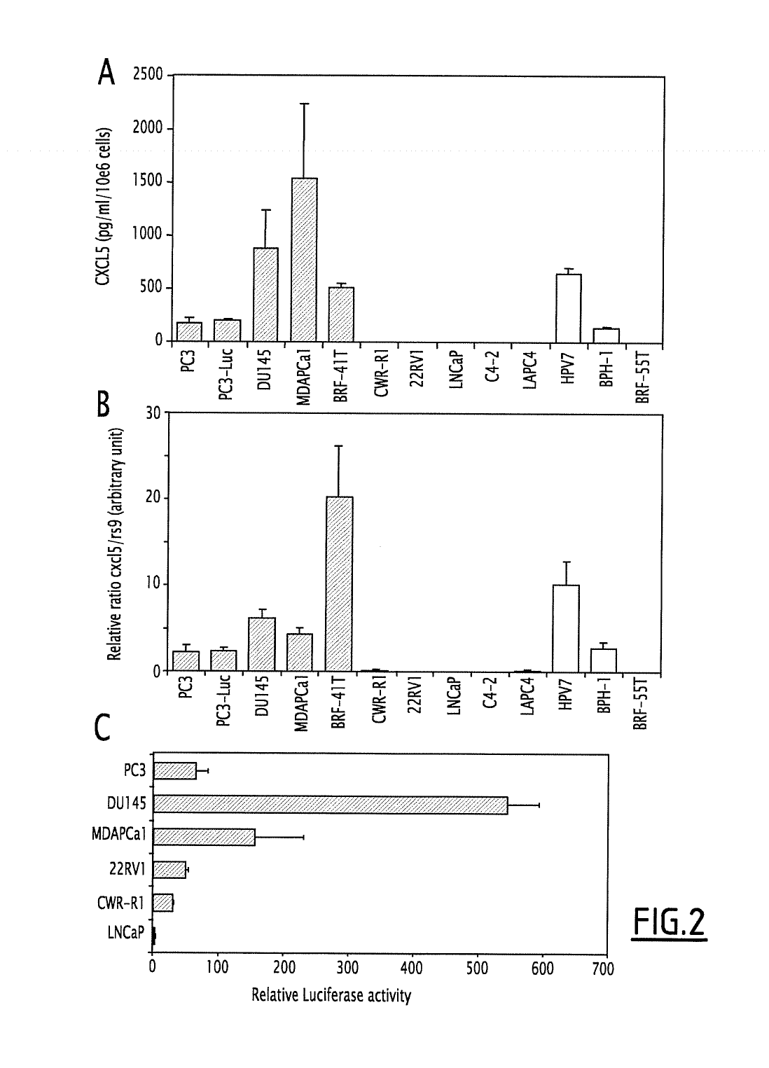

AR-negative aggressive cell lines secreted higher levels of CXCL5 (FIG. 2A) compared to AR-positive cell lines. In addition, hyperplasic cell lines secreted very variable levels of CXCL5.

These results obtained through an ELISA assay were confirmed at the RNA level (FIG. 2B).

Finally, a CXCL5 promoter construct was transfected into different prostate cancer lines. It was shown that CXCL5 promoter activity was higher in AR-negative prostate cancer lines compared to AR-positive cell lines (FIG. 2C).

Taken together, these results demonstrate that CXCL5 is highly expressed by epithelial cells of high grade prostate tumors, i.e. in androgen-independent prostate cancer cells.

### Example 3

### CXCL5 Enhances the In Vitro Growth of Prostate Cancer Cells

Stable transfectants of CWR-R1 cells were generated by transfection with CXCL5 cDNA. The stable CWR-R1 transfectants thus obtained were further transfected with the luciferase gene in order to monitor their growth in vivo. Wild-type CWR-R1 cells (referred to as CWR-R1) and CWR-R1 cells transfected only with the luciferase gene (referred to as CWR-R1Luc) were used as controls.

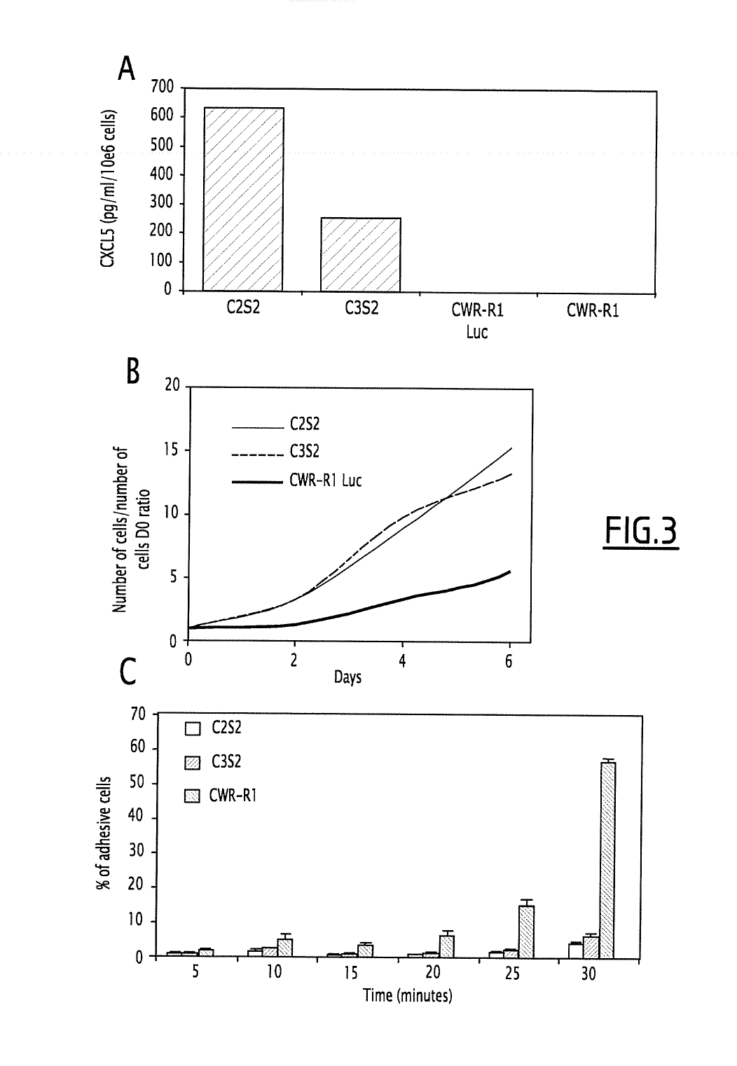

Two distinct clones, referred to as C3S2 and C2S2, were selected. These clones exhibited moderate (C3S2) and high (C2S2) expression of CXCL5 respectively (FIG. 3A). Of particular note, CXCL5 secretion in these clones was lower in AR-negative cell lines such as DU-145 or MDA-PCa1.

CXCL5 expressing clones displayed a growth that was a two to three rapider than parental CWR-R1 cells (FIG. 3B).

Since tumor cells must have an altered ability to attach to the support in order to metastasize, a kinetic of in vitro attachment to plastic dishes was carried out. It was shown that C2S2 and C3S2 clones had a slower kinetic of attachment than wild-type cells (FIG. 3C).

Taken together, these results demonstrate that transfection of normal cells with CXCL5 leads to the obtention of transfectants that exhibit a rapider growth and an altered ability to attach to the support compared to wild-type cells.

### Example 4

### CXCL5 Dramatically Increases In Vivo Proliferation of Prostate Cancer Cells

The effect of CXCL5 on tumor growth was then evaluated.

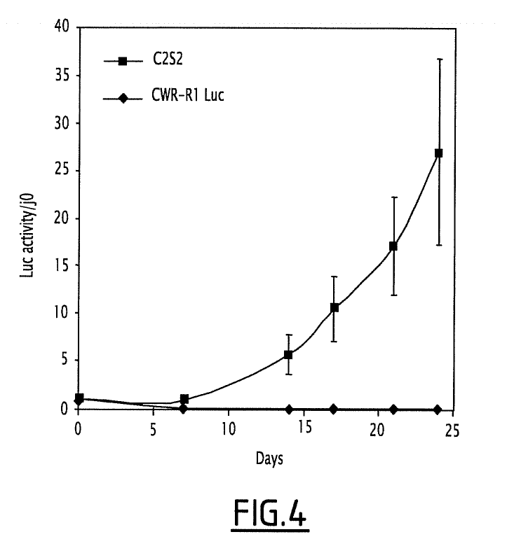

CWR-R1-LUC cells or C2S2 cells were injected in the prostate of athymic mice. Interestingly, cells expressing CXCL5 displayed a dramatic increase of their growth compared to wild-type cells (FIG. 4).

This result further demonstrates that CXCL5 plays a role in cell proliferation.

### Example 5

### CXCL5 is an Androgen-Regulated Gene

To assess whether CXCL5 could be involved in hormone escape of prostate tumors, it was next studied whether CXCL5 was regulated by androgens.

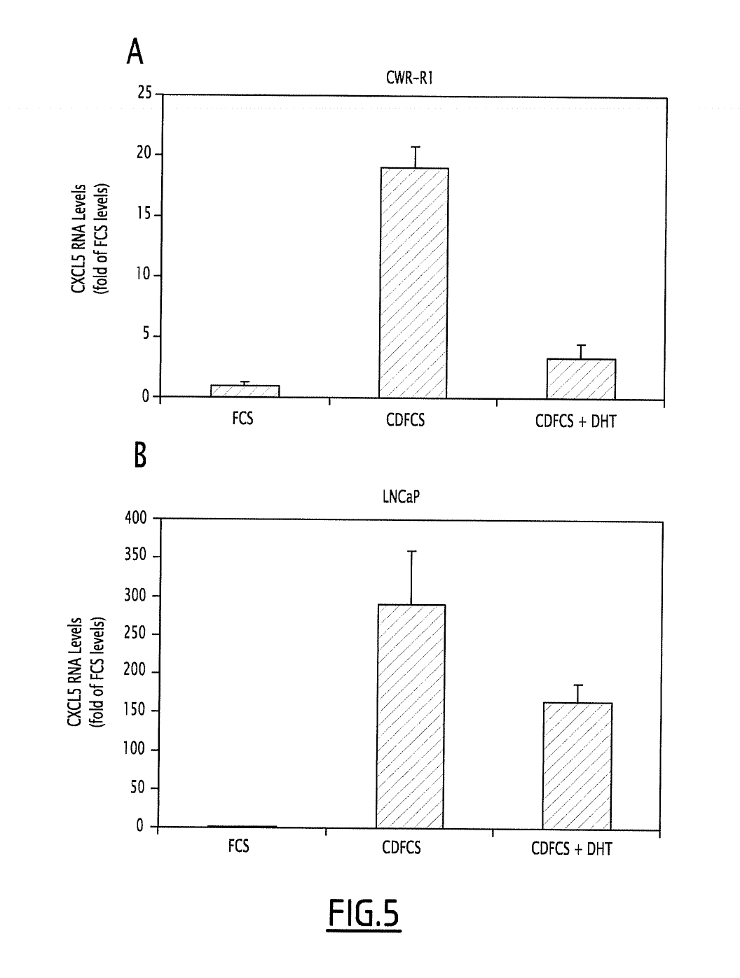

It was observed that elimination of androgens by desteroidation of the serum strongly increased CXCL5 RNA levels in CWR-R1 (FIG. 5A) and LNCaP (FIG. 5B) cells. On the other hand, addition of dihydrotestosterone to the medium partially reduced levels of CXCL5 RNA (FIG. 5).

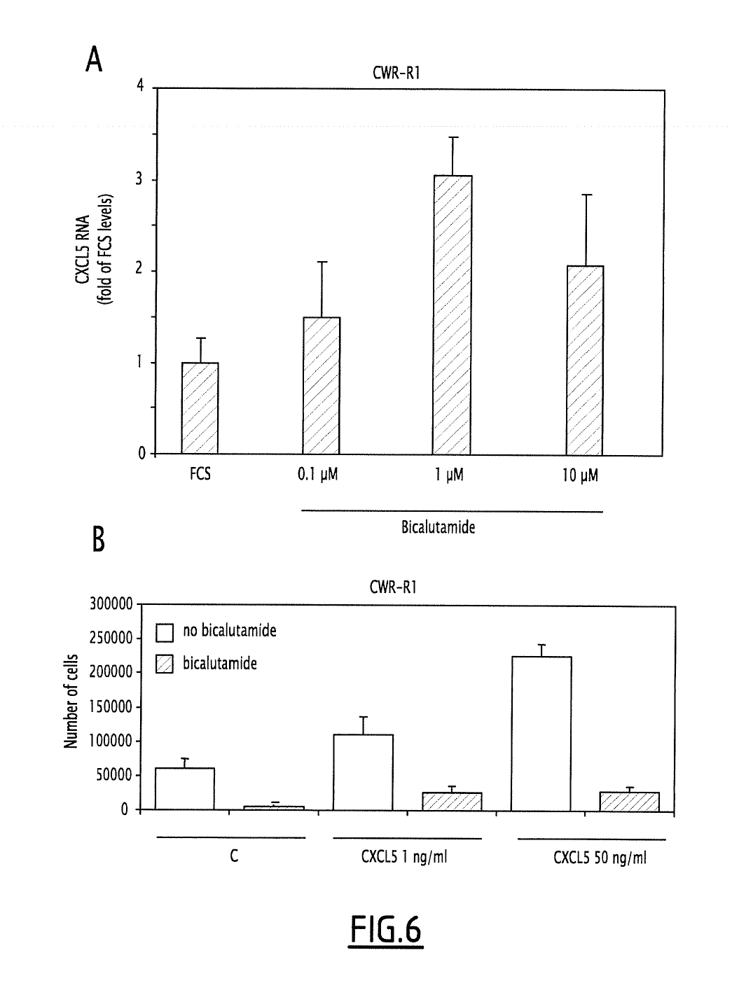

The effects of bicalutamide (an anti-androgen) on CXCL5 expression were also studied. It was observed that bicatulamide increased CXCL5 RNA by two to three fold in CWR-R1 cells (FIG. 6A).

CWR-R1 cells treated with bicalutamide displayed a reduced proliferation (FIG. 6B). On the contrary, as observed for stable clones expressing CXCL5, addition of recombinant CXCL5 increased two to four fold the proliferation of CWR-R1 cells (FIG. 6B). Interestingly, CXCL5 addition could also partially alleviate the inhibition of proliferation by bicalutamide (FIG. 6B).

This result shows that CXCL5 is negatively regulated by androgens.

### Example 6

### CXCL5 Increases the Proliferation of CWR-R1 Cells Both in the Absence and in the Presence of Bicalutamide

It was next determined whether CXCL5 could confer an androgen-independent growth to CWR-R1 cells in vivo.

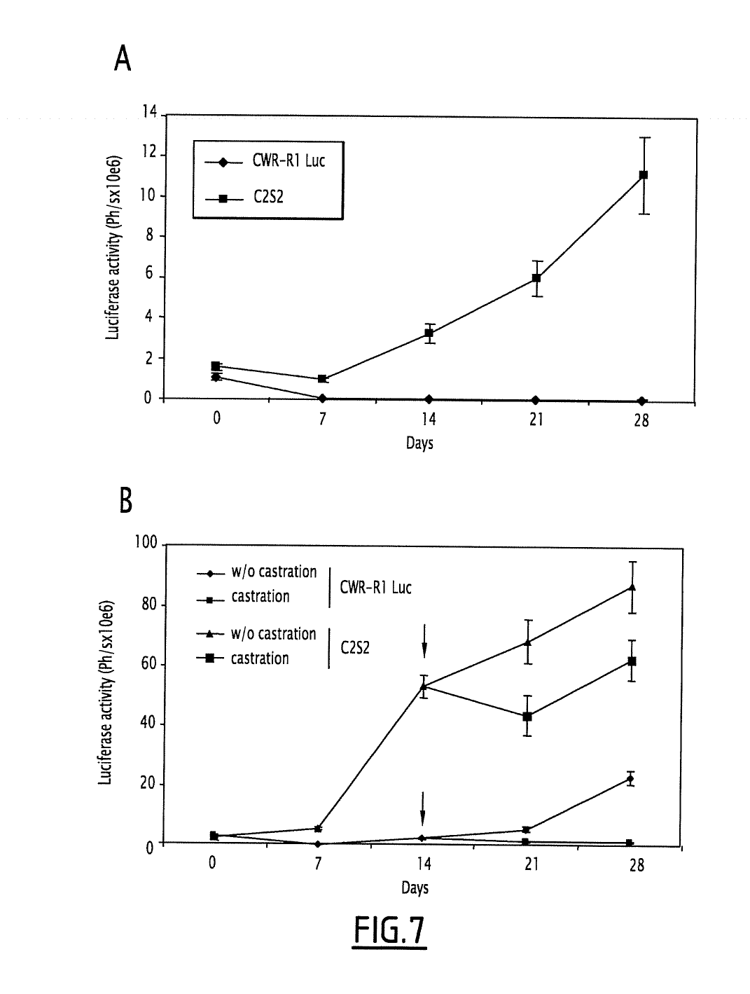

Athymic mice were castrated, and then CWR-R1 or C2S2 cells were inoculated in the prostate (FIG. 7A). CWR-R1 cells were unable to form tumors in the absence of androgens. On the other hand, C2S2 cells could form tumors very rapidly in castrated animals.

To evaluate hormone escape, CWR-R1 or C2S2 cells were injected in the prostate of athymic mice (FIG. 7B). Two weeks after inoculation, mice were divided in two groups, one which was castrated, and the other which was sham operated. CWR-R1 cells could not grow in castrated animals, but could grow in intact animals, even though with a two to three weeks delay compared to C2S2 cells (FIG. 7B). On the other hand, C2S2 cells were weakly affected by castration, and after a week of decreased growth, grew again as rapidly as cells present in non castrated animals.

These result show that when animals bearing tumors expressing CXCL5 are castrated, hormone escape arises. In contrast to this, wild-type cells fail to escape. Overall, these data show that CXCL5 is a novel androgen-regulated gene involved in hormone escape of prostate cancer cells.

### Example 7

### Seric CXCL5 Levels are Correlated to Intra-Tumor Expression of CXCL5

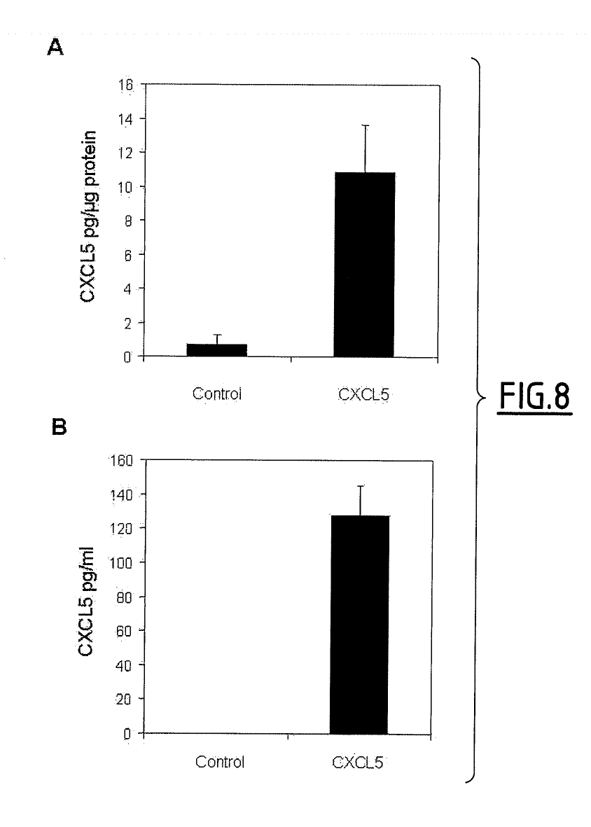

To determine whether the measure of seric concentration could be feasible and in agreement with intra-tumor expression of CXCL5, the expression of CXCL5 in the serum and in whole cell extracts of tumor of mice bearing either CWR-R1 or CWR-R1-CXCL5 tumors was analyzed. As shown on FIG. 8A, intra-tumor expression was more than 10 fold higher in tumors constituted of CWR-R1-CXCL5 cells than in tumors of CWR-R1 cells. In the serum of animals bearing CWR-R1 tumors, we could not detect CXCL5 levels, whereas, CXCL5 levels were high (about 130 pg/ml) in animals bearing CWR-R1-CXCL5 tumors (FIG. 8B). This demonstrates that seric CXCL5 levels are in agreement with the ones of tumor levels.

### Example 8

### Inhibition of CXCL5 Action Reduces Cell Proliferation

Based on the previous results, blocking CXCL5 action could constitute a promising therapeutical approach. To demonstrate the rationale of this, two approaches were used, one blocking CXCL5 action by using specific blocking antibodies, the other one, by down-regulating CXCL5 expression with a shRNA method.

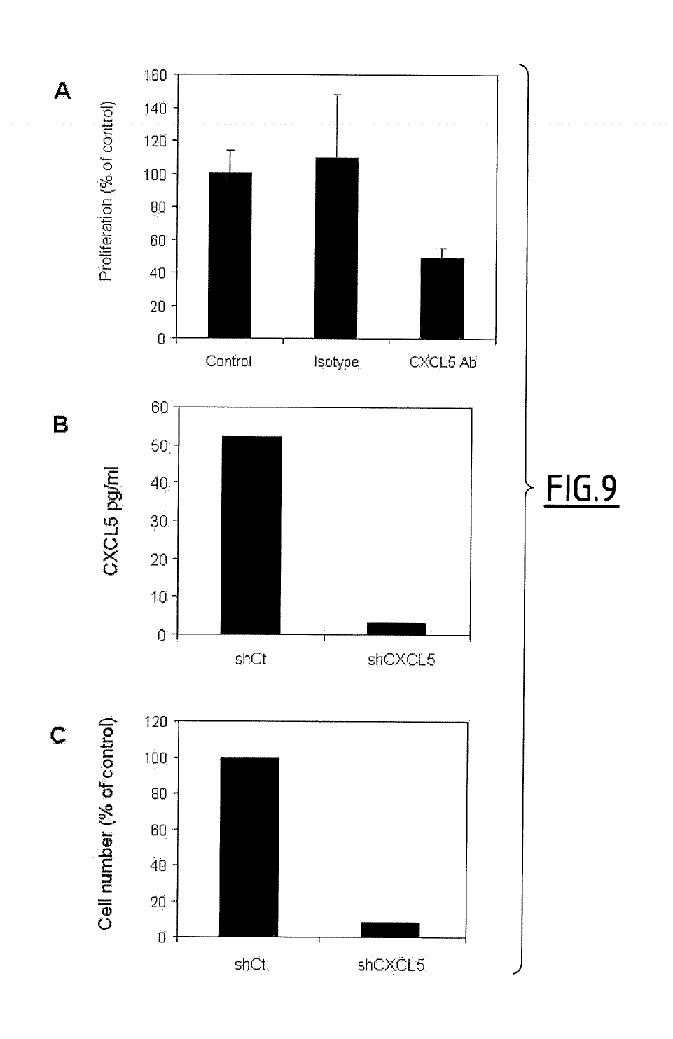

PC-3 cells which naturally express high levels of CXCL5 and are highly aggressive were used. When blocking CXCL5 action with a specific antibody, it was observed that the proliferation of PC-3 cells was strongly reduced compared to non specific immune serum (FIG. 9A).

CXCL5 expression in PC-3 cells was inhibited by transfecting them with a shRNA constructs against CXCL5. It was observed that CXCL5 expression was strongly reduced compared to the Non-Target shRNA Control Vector after 4 days of inhibition (FIG. 9B). When measuring the proliferation of the transfected cells, it was observed that CXCL5 inhibition could also dramatically inhibit the proliferation of PC-3 cells by more than 10 fold.

In conclusion, the two approaches clearly demonstrate that blocking CXCL5 action can strongly reduce its mitogenic effects.

### Example 9

### CXCL5 can be Detected in Human Serum and Urine

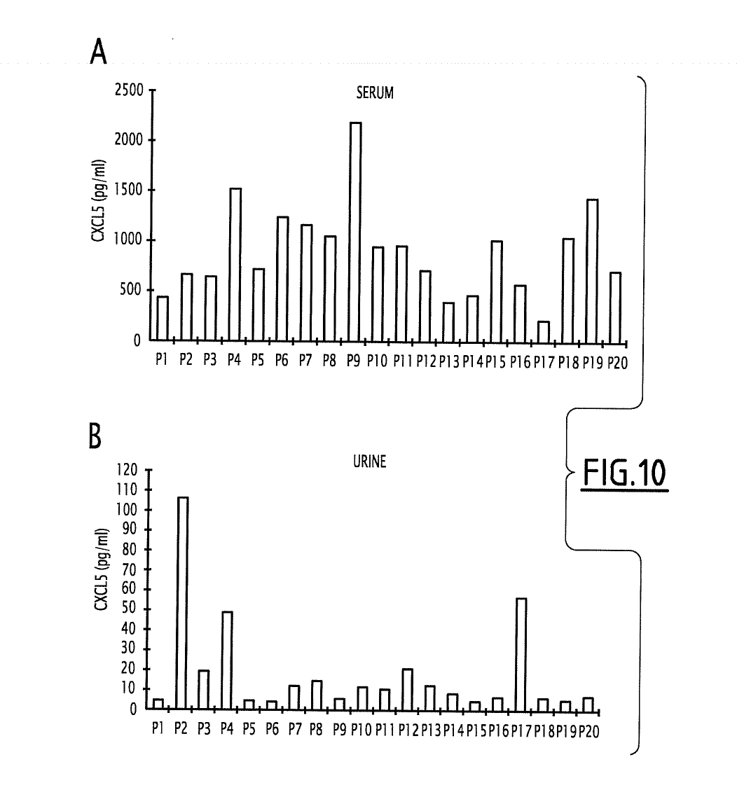

The goal of this experiment was to demonstrate that CXCL5 protein could be detected in the blood and the urine of human patients. To achieve this, the blood and urine of 20 patients with a suspicion of prostate cancer were collected. We settled a sensitive chemiluminescent ELISA assay (see paragraph 1.15 of Example 1). In the serum, CXCL5 levels ranged from 217 pg/ml to 2182 pg/ml. In the urine, the levels of CXCL5 were much lower and ranged from 4.2 pg/ml to 57 pg/ml (FIG. 10). This demonstrates that CXCL5 protein can be detected in the urine and serum of patients.

### Example 10

### Conclusion

Hormone escape is the main issue in prostate cancer treatment, as it arises for all patients treated with anti-androgens after a certain time ranging from 14 to thirty months. The search of markers and therapeutic targets for hormone-refractory prostate cancers appears crucial. We show here that the chemokine CXCL5 (ENA-78) is highly expressed by epithelial cells of high grade prostate tumors. Moreover, invasive prostate cancer cell lines produce higher levels of CXCL5 compared to less aggressive cell lines. This higher expression is due to a higher transcriptional activity of CXCL5 gene promoter. Stable transfection of CXCL5 cDNA in the androgen-receptor (AR)-positive prostate cancer cell line CWR-R1 increases its in vitro growth and reduces its ability to adhere to plastic dishes. In addition, CXCL5 confers to these cells a spectacular growth in vivo. We also demonstrate that CXCL5 gene is androgen-regulated: removal of androgen up-regulates its expression, whereas addition of androgens reduces its expression. In addition, treatment of CWR-R1 cells with the anti-androgen bicalutamide increases CXCL5 expression. CXCL5 treatment of CWR-R1 cells is able to increases in vitro cell growth even in the presence of bicalutamide. In vivo, CXCL5 enables tumor take of CWR-R1 cells in the absence of androgens, whereas wild-type cells fail to grow in these conditions. Moreover, when animals bearing CWR-R1-CXCL5 tumors are castrated, hormonal escape arises, whereas wild-type cells fail to escape. We also show that CXCL5 seric levels are correlated to intra-tumor expression of CXCL5 in mice. In humans, CXCL5 could be detected in the serum and urine. Moreover, we show that blocking CXCL5 action by CXCL5 blocking antibody or by down-regulating its expression with a siRNA approach inhibits prostate cancer cell proliferation. Overall, our data show that CXCL5 is a novel androgen-regulated gene involved in hormone escape of prostate cancer cells, that could constitute a novel therapeutic target.

1. (canceled)

2. The method of claim 16, wherein an increase of at least two-fold indicates that prostate cancer cells are undergoing hormone escape.

3. The method according to claim 16, further comprising the step of designing a treatment regimen for said individual based on results obtained in said concluding step.

4. The method of claim 16, wherein steps (a)-(d) are repeated at least at two different points in time in order to monitor the onset of hormone escape in said patient and/or to monitor responsiveness of said patient to a drug.

5. The method claim 16, wherein measuring step (b) is performed by one or more of immunochemistry, Elisa or reverse transcription polymerase chain reaction (RT-PCR).

6. The method according to claim 16, wherein said biological sample is selected from the group consisting of prostate tissue, prostate cells, serum and urine.

7-15. (canceled)

16. An in vitro method for assessing hormone escape in a patient suffering from prostate cancer, comprising the steps of:
a) obtaining a biological sample from said patient;
b) measuring
i) the amount of CXCL5 protein in said biological sample using a technique elected from the group consisting of immunochemistry, Elisa, Western blotting, and flow cytometry, and/or
ii) the level of CXCL5 mRNA in said biological sample using a technique selected from the group consisting of Northern blotting, polymerase chain reaction (PCR), ligase chain reaction (LCR), transcription-mediated amplification (TMA), strand displacement amplification (SDA) and nucleic acid sequence based amplification (NASBA);
c) comparing the amount of CXCL5 protein and/or the level of CXCL5 mRNA measured at step (b) with the amount of CXCL5 protein and/or the level of CXCL5 mRNA measured in a negative control sample; and
d) if an increase is detected in the amount of CXCL5 protein and/or the level of CXCL5 mRNA in said biological sample compared with the amount of CXCL5 protein and/or the level of CXCL5 mRNA in said negative control, then concluding that prostate cancer cells in said patient are undergoing hormone escape; or
if an increase is not detected in the amount of CXCL5 protein and/or the level of CXCL5 mRNA in said biological sample compared with the amount of CXCL5 protein and/or the level of CXCL5 mRNA in said negative control, then concluding that prostate cancer cells in said patient are not undergoing hormone escape.

17. The method of claim 16, wherein said step of measuring an amount of CXCL5 protein is performed using antibodies specific for detecting CXCL5 protein.

18. The method of claim 16, wherein said step of measuring a level of CXCL5 mRNA is performed using probes or primers specific for detecting CXCL5 mRNA.

19. The method of claim 16, further comprising the steps of detecting and analyzing the amount and/or expression level of CXCL8 in said biological sample.
